(19) United States
(12) Patent Application Publication
(10) Pub. No.: US 2008/0251313 A1
(43) Pub. Date: Oct. 16, 2008

(54) Disposable cover for stethoscope head

(76) Inventors: JoAnn F. Knight, San Francisco, CA (US); Carl E. Knight, San Francisco, CA (US)

Correspondence Address: JoAnn F. Knight, 219 6th Street, San Francisco, CA, 94103, US

(21) Appl. No.: 11/785,350

(22) Filed: Apr. 16, 2007

(51) Int. Cl. A61B 7/02

(52) U.S. Cl. 181/131

(57) ABSTRACT

A disposable cover is provided for a stethoscope head, the stethoscope stem and the tubing immediately adjacent to the stem to help provide a barrier to prevent the transmission of harmful organisms or matter from the stethoscope head to a patient, or to the medical practioner, or to the general public and vice versa. The cover has an opening which is stretched to receive the stethoscope head and a loose casing capable of enveloping the stethoscope head. The loose casing does not need to stretch during insertion or while enveloping the stethoscope head. The loose casing allows the stethoscope head to simply drop down into the cover to the closed end.

### FIELD OF THE INVENTION

The present invention relates to a practical, low-cost disposable cover for a stethoscope head to help prevent the transmission of harmful organisms both to and from the stethoscope head.

### FIELD OF SEARCH

181#131 and 428 #192

### REFERENCES CITED

| AP No. 20040091678 | Jordan | May 4, 2004 |
|---|---|---|
| AP No. 20040048539 | Moncion | March, 2004 |
| Pat No. 6467568 | Kemper | October, 2002 |
| 6206134 | Stark | March, 2001 |
| 5747751 | Weckerle et al | October, 1996 |
| 5269314 | Kendall et al | December, 1993 |
| 4871046 | Turner | October, 1989 |
| 4867268 | Ulert | September, 1989 |
| 4401125 | Taylor et al | August, 1983 |
| 3225841 | Hasbrouch | June, 1966 |

### PRIOR ART

Previous attempts have been made to create a protective cover for a stethoscope head. Weckerle, U.S. Pat. No. 5,747,751 and Kemper, U.S. Pat. No. 6,467,568 effectively rebut a variety of such devices including

“Disposable Stethoscope Head Shield” of Turner

U.S. Pat. No. 4,871,046

“Sanitary Stethoscope” of Ulert

U.S. Pat. No. 4,867,268

“Stethoscope Bell Cover” of Hasbrouch

U.S. Pat. No. 3,225,841

“Stethoscope Securing Pad” of Taylor et al

U.S. Pat. No. 4,401,125

“Stethoscope Cover” of Kendall et al

U.S. Pat. No. 5,269,314

“Stethoscope Cover” of Stark

U.S. Pat. No. 6,206,134

Each of these devices does leave much of the stethoscope head uncovered or else is difficult to use.

Some devices including Moncion's AP No. 20040048539 are designed for ornamental use on a stethoscope head but not for use as a barrier against the transmission of harmful organisms. Mancion's cover would not be able to function as a barrier because the knitted materials suggested for ornamentation are porous by nature. In contrast, my cover is primarily for use as a barrier against the transmission of harmful organisms or matter, and is formed from non-permeable material.

The device/cover of Jordan, AP 20040091678 has as its focus a universal cover with a metallic foil rim not proposed for use as a stethoscope cover. The foil rim would be abrasive against the stethoscope diaphragm and interfere with the sound transmitting quality of the stethoscope's diaphragm. In contrast, my cover is made of acoustically sound-transmitting material that does not interfere with the quality of sound transmitted by the stethoscope's diaphragm.

Weckerle's et al, U.S. Pat. No. 5,747,751, cover is difficult to put over the stethoscope head without having the user reach into the cover in order to stretch the smaller intermediate neck section to a width able to accommodate the insertion of the stethoscope head. Obviously, the stethoscope head would come in contact with the user's fingers for the length of the entire neck area of the cover as it is held stretched open for the passing stethoscope head. In direct contrast, the cover of my present invention needs only the rolled hem edge opening to be stretched in order to allow insertion of the stethoscope head into the casing. The user holds only the stethoscope tubing and adjacent stem to control the stethoscope head as it passes into the casing. The stethoscope head need never touch the user's finger as the fingers stretch the rolled hem edge opening. Once past the rolled hem edge opening gravity allows the stethoscope head to drop right into the loose fitting case. It is very important that the stethoscope head is not touched by the medical practioner as the stethoscope head is inserted into the cover. The only way to stop the circle of transmission of disease is to eliminate contact with the immediate surfaces of the stethoscope head. My cover provides for this primary, essential need because of the cover's unique design which is contrary to Weckerle's because my cover has no smaller neck section. Weckerle's cover is not able to meet this essential element of design because the smaller intermediate neck section will never fit over the stethoscope head unless the neck section is held open during insertion of the stethoscope head.

Further, when removing Weckerle's stretched, tight fitting cover from the stethoscope head, the cover must be peeled off the stethoscope head; or else the cover must be rolled over itself across the diaphragm and bell portion of the stethoscope head thus exposing the interior of the casing, which has just been in contact with the stethoscope head, to the user. Vice versa, the stethoscope head is in contact with the outside of the casing which has just been in contact with the patient.

The present invention provides a stethoscope cover in direct contrast to Weckerle's. Due to the loose casing which is easily removed the present invention allows the user to use only a slight pull to release the rolled hem edge of the cover from the stethoscope head as the cover is removed. My cover does not need to be rolled over itself exposing the interior of the casing as it is removed.

It is futile to use any method or design for a stethoscope cover which method does not prevent contamination of the stethoscope head in the first place. It is also futile to cover the stethoscope head before using it and then expose the potentially contaminated interior of the casing to the user. Every time the stethoscope head comes in contact with a user's hand, a patient's skin, or in contact with any contaminated surface, there is an exchange of organisms between the two surfaces. My cover of this present invention will help prevent the exchange of organisms from surfaces to the stethoscope head; and from stethoscope head to other surfaces.

In practical use, the cover of the present invention would be placed on a clean, uncontaminated stethoscope head before the stethoscope is used on any patient. My disposable cover would be removed after the patient examination. The stethoscope, stem and adjacent tubing would not be contaminated during the removal of my cover. The stethoscope head, stem and tubing are protected from contamination while the cover is properly over the stethoscope head. The present invention in long enough, and wide enough to cover most commonly used stethoscope heads on the market.

Once the disposable cover of this invention is removed from the stethoscope head, another clean, disposable cover should be immediately put over the stethoscope head if the stethoscope could possibly be contaminated during non-use. Then, before use a new, clean cover would be used over the stethoscope head just prior to patient examination.

Considering the volume of covers required to keep the circle of disease transmission closed to contamination, the use of biodegradable materials to form stethoscope covers is of utmost concern. The present invention provides a loose fitting casing which overcomes the potential for easy tearing of the thinner, bio-degradable materials as the cover is used.

All of the above expressed needs are provided for in my present invention and are not met by the prior art.

### BACKGROUND OF THE INVENTION

Transmission of disease in a public concern. Public research and the Center For Disease Control all call attention for the need to reduce exposure to harmful organisms and bacterias. The need to specifically reduce the transmission of these harmful organisms or matter via the stethoscope is well documented. Weckerle et al U.S. Pat. No. 5,747,751 has pointed out the research well.

Most concern has been centered on contamination of the stethoscope during patient examination, and vice versa, the contamination of the patient via the stethoscope. Thus, the transmission of disease continues on patient to patient and even patient to the practioner via the fouled stethoscope. Of equal importance however, is contamination of the stethoscope from daily exposure to harmful organisms or matter when the stethoscope is not in actual use during a patient examination. It is amazing that little if any fear of stethoscope contamination has been noted when the stethoscope is carried around the neck by medical persons almost as a status symbol. Carried thus the stethoscope goes from home to office, to the back seat of cars (where it is soiled by dog or cat hair, fecal matter etc.). The stethoscope appears in public restrooms, and on public transportation. It is stored in lockers right along side shoes or dirty laundry. The stethoscope is observed in all these public places, where it is hopelessly contaminated and then brought casually to the patient for exams.

On top of all that, the patient with harmful organisms or drug-resistant antibiotic disease, adds to the contamination of the stethoscope. Then, at the end of the day, the stethoscope makes its rounds in the public domain to spread the harmful organisms and disease while even more public contamination continues. The crisscross transfer of harmful organisms is utterly endless. Now that the stethoscope is seen as a status symbol it is the vector of disease second only to the physician's proud, bloody coats of Dr. Semilweis' day.

It is common knowledge that the stethoscope can be a potential vector of hospital acquired infection. Antibiotic-resistant organisms are on the rise. Reference can be made to Jeffrey Jones, M. D. FACEP in his book Annals of Emergency Medicine Chapter 26, 3 September 1999 pages 296-299

### “Stethoscope: A Potential Vector of Infection?”

Weckerle, U.S. Pat. No. 5,747,751 accurately cites this reference and others which prove the need for a disposable stethoscope cover. Requests for bio-degradable products are on the rise. My cover design allows for such thinner, biodegradable materials to be used because the cover casing itself is not subject to the stress of stretching during insertion, use, or removal of the stethoscope head and the casing. Stretching of the biodegradable material could more easily result in a tear or break in the cover compared to material such as Latex or vinyl. Tears allow for the transmission of harmful organisms.

Obviously, the need to cover and protect the stethoscope from contamination is real. The need to protect the practioner, the patient and the public from the stethoscope and its potential harmful organisms is real.

To date there is no disposable stethoscope cover readily available on the market. Once such a cover is available it should be only a matter of time before the patient, the public, the practioner, insurance carriers, and hopefully the Center For Disease Control demand the use of the cover.

It is the purpose of this present invention to provide such a disposable stethoscope cover.

### SUMMARY OF THE INVENTION

The present invention is a disposable cover for a stethoscope head and the immediately adjacent stethoscope tubing.

The cover is made of material suitable to form a barrier against harmful organisms or matter that could be transmitted to a patient.

The cover is acoustically transmissive so as to not interfere with the normal sound receiving diaphragm of the stethoscope head. The cover has a stretchable rolled hem which is smaller in width than the stethoscope head; and a casing whose entire width from just below the rolled hem to the closed, seamless end is wider than the stethoscope head.

In an unstretched position the rolled hem helps to hold the wider stethoscope head inside the casing. The casing lays flat against the stethoscope diaphragm as a natural result of the flexible material of the cover. The loose fit of the cover around the stethoscope head allows for easy introduction of the head into the casing with no contact ever necessary between 1) the practioner and the stethoscope head; 2) the stethoscope and the outside of the disposable cover 3) the patient and the stethoscope head.

The cover protects the stethoscope head from contaminating a patient and spreading harmful organisms. The cover protects the stethoscope head from the contamination of harmful organism already on a patient, thus reducing the circle of transmission of disease from patient to patient; or disease from patient to practioner, which occurs when an uncovered, soiled stethoscope head is allowed to transfer soiled material to a new individual. Using a clean cover protects the stethoscope during periods of time when the stethoscope is not actually in use but is subject to exposure in the general environment to potentially harmful materials, matter, or organisms. A new, clean cover can easily be replaced every time a patient is to be examined.

The cover of the present invention could be made from latex, vinyl, and other non permeable, even biodegradable materials. The natural static attraction of these materials to the stethoscope diaphragm helps to keep the cover in place, flat against the stethoscope head during patient examination. This natural attraction in no way inhibits the very easy motion of sliding the cover off the stethoscope head. This is important because the removal of the cover must be as smooth as possible to avoid tears or breaks in the casing. The present invention provides a loose casing to avoid stressing of the material at any time during use.

It is the object of this invention to provide a cover for a stethoscope head in order to assist in the prevention of the transmission of harmful organisms both to the stethoscope head and by the stethoscope head.

It is the further object of this invention that it is easy to put on, easy to take off, requires minimal pulling or forceful stretching of the cover as it is used in order to reduce accidental tearing of cover, and subsequent contamination of a patient.

It is further the object of this invention to provide a design for a stethoscope cover that will allow the practical use of bio-degradable materials to form the disposable cover.

Further, in the interest of providing a lower cost cover for a stethoscope head the present invention can be made in one uniform width from open end to the closed end. A width wider than the width of a stethoscope head beginning at the open end and continuing through-out the depth of the cover to the closed end would provide a cover able to fit quickly over stethoscope head while avoiding any stretching of the open end when the stethoscope head is inserted into the cover. If the cover of the present invention were made in this wide uniform manner it would be possible to avoid the costs associated with the more precise tooling required to produce the cover with the rolled hem edge.

The rolled hem edge is necessary when stretching of the open end occurs during insertion of the stethoscope head into cover that is not wider than the width of the stethoscope head. The main drawback to a wide uniform cover is the potential for the stethoscope head to slip easily out of the wide cover. However, because the cover of the present invention is long enough to cover the stethoscope head and adjacent tubing the medical practioner's hand holds the cover in place as the stethoscope is gripped in its commonly used manner. Thus, the problem of a cover possibly coming off the stethoscope head unexpectedly is easily and naturally solved in common, known manner. In the interest of possibly saving money in the manufacturing process the uniform wide width cover has merit.

### BRIEF DESCRIPTION OF THE DRAWINGS

FIG. 1 is a top perspective view of a disposable cover for a stethoscope head and adjacent tubing

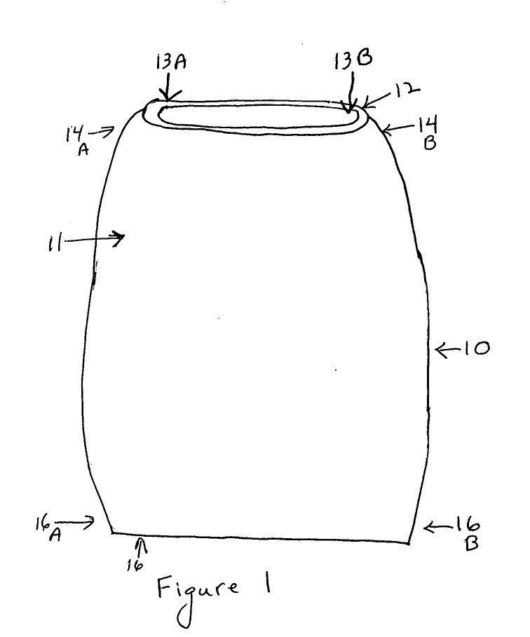

FIG. 2 is a top perspective view of a disposable cover with a stethoscope head illustrated by dashed lines shown inserted into the cover, The drawing is to scale and illustrates the loose fitting casing of the cover.

10 Disposable cover
11 casing
12 rolled hem of the receiving end
13 A [symbol] B width of rolled hem edge
14 A [symbol] B width of the receiving end for comparison to the width for of the rolled hem edge of the receiving end
16 close end
16 A [symbol] B width of the smooth, seamless closed end for comparison to width of the stethoscope head and the width of the receiving end
20 stethoscope
22 stethoscope head
23 stethoscope diaphragm
24 stethoscope bell portion
25 stethoscope stem
26 stethoscope tubing

### DETAILED DESCRIPTION OF THE INVENTION AND PREFERRED EMBODIMENT

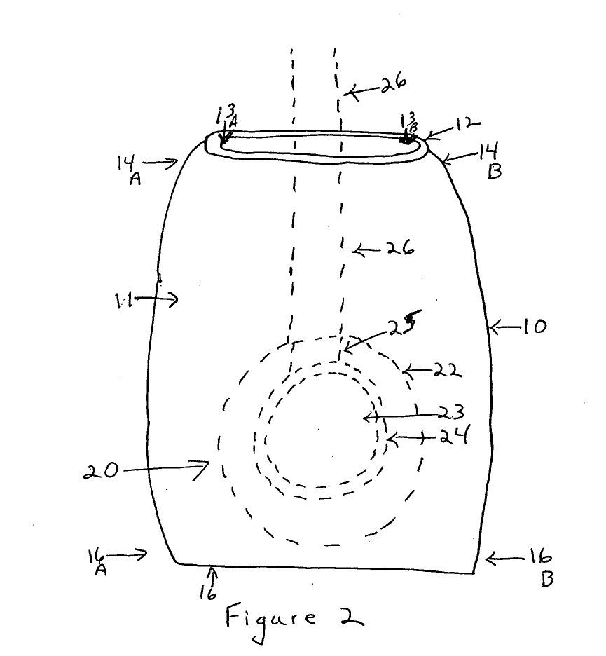

Referring to the figures, disposable cover 10 of the invention is generally configured as a smooth, flexible, seamless casing for receiving, covering and retaining a stethoscope head. Stethoscope 20 shown in dashed lines in FIG. 2 comprises stethoscope head 22 and stem 25 extending from stethoscope head 22 to connect stethoscope head 22 to stethoscope tubing 26. Tubing 26 extends from stethoscope head 22 to ear pieces, not shown. Stethoscope head comprises diaphragm portion 23 and bell portion 24 common to most currently used stethoscopes. The disposable cover 10 will accommodate single or double-headed stethoscopes due to the loose configuration of the casing 10 and the stretchable, rolled hem 12 of the invention. In my current invention it will be seen that only the rolled hem edge 12 needs to be stretchable. Some flexibility of cover 10 below the rolled hem edge 12 is natural to the cover 10 due to the materials used in the medical industry to prevent transmission of harmful organisms. Such materials as latex or vinyl are commonly used. However, the disposable cover 10 of my invention is not dependent on a cover 10 which stretches as it envelopes the stethoscope head 22. As shown in FIG. 2 cover 10 is wider from points 14A to 14B than stethoscope head 22. FIG. 2 also shows that the closed end of cover 10 from points 16A to 16B is wider than the stethoscope head 22. This width of cover 10 allows for very easy insertion of stethoscope head 22 into cover 10 because the only snug fitting around the stethoscope head 22 is the rolled hem edge 12. In the preferred embodiment of my present invention the disposal cover 10 fits so loosely around the stethoscope head 22 that the medical user need never to reach beyond the rolled hem edge 12 in order to stretch the cover 10 and insert stethoscope head 22. Also, the medical user is able to hold only the tubing 26 to control stethoscope head 22 during insertion of stethoscope head 22 into cover 10. This feature of the present invention in crucial to avoid the circle of contamination which begins with touching at any time the contaminated stethoscope head 22.

In the present invention the cover 10 fits loosely enough around the stethoscope head 22 that minimal effort is required to release the cover 10 from stethoscope head 22. The less manipulation of the cover 10 the more apt the cover 10 is to remain without tears or breaks.

In the preferred embodiment, the stethoscope head 22 is retained in the disposable cover 10 because of the width of rolled hem edge 12 and not because the cover 10 is snug around stethoscope head 22.

The loose fitting design of cover 10 avoids the need to ever stretch the cover 10 below the rolled hem edge 12. Rolled hem edge 12 can be formed to sufficiently reinforce the opening of cover 10 even as the reinforcing rolled hem edge 12 maintains it stretch. Thus, strength and stretch are achieved where needed in the present invention. Loose fit of the cover 10 around stethoscope head 22 is an asset to the ease of use of the design of my disposable stethoscope cover 10.

The disposable stethoscope cover 10 can be of colored material to alert to special medical issues. For instance a cover 10 red in color could indicate use of a stethoscope 20 in an environment known to be contaminated with drug-resistant bacteria. The color red would easily identify a stethoscope cover 10 as potentially needing extra caution to avoid cross infection.

My disposal cover 10 can be detailed with a name or personal design so that a lost stethoscope 20 could be returned to the owner.

My cover can be personally ascribed to a patient during extended patient stay in a hospital. The patient's name could be written on the cover 10 so that the stethoscope 20 remains in the room as a dedicated part of that patient's care until his release. This particular use would result in less need for disposable covers 10 but still control potential transmission of harmful organisms or matter.

The cover 10 of the present invention can be applied with anti-microbial sprays or powders to enhance its effectiveness in extreme environments known to contain drug-resistant organisms.

My disposable cover 10 can be decorated so as to appeal to children who are frequently fearful of all medical equipment.

In a second embodiment of the disposable stethoscope head cover 10 the rolled hem edge 12 as presented in the first embodiment of the cover 10 is eliminated and the open receiving end 14 is retained wider than the width of a stethoscope head 22. The cover 10 is wider than the width of a stethoscope head 22 from the open receiving end 14 through the depth of the easing 11 to the closed end 16

The wider open receiving end 14 allows for a stethoscope cover 10 that never needs to be stretched at the open receiving end 14 as the stethoscope head 22 is inserted into the cover 10. This is a valuable point because many environmentally friendly, biodegradable materials are flexible but relatively thin compared to other non-bio-degradable materials.

This wider open receiving end 14 of the disposable cover 10 provides for the use of bio-degradable materials.

Every enhanced feature of the design of this present invention is put forth to provide a low cost, simple to use disposable stethoscope cover 10 to help prevent the transmission of disease, harmful organisms and harmful matter.

What is claimed is:

1. A disposable cover for a stethoscope head and the tubing immediately adjacent to said stethoscope head comprising:
(a) a seamless casing formed from a sound transmitting material which is substantially capable of preventing the transmission of harmful organisms and matter.
(b) said seamless casing having an open end of a dimension when not stretched that is smaller than the width of the stethoscope head.
(c) said open end comprised of a hem edge sufficiently rolled to a thickness to re-enforce said open end
(d) said open end comprised of rolled hem edge capable of stretching to allow insertion of a stethoscope head into the casing.
(e) said casing having a width when not stretched greater than the stethoscope head.
(f) said casing longer than the length of the stethoscope head, stem and adjacent tubing

2. The disposable cover of claim one wherein said casing is formed from material that is anti-microbial.

3. The disposable cover of claim one, wherein said casing is formed from material to which anti-microbial sprays or powders may be applied.

4. The disposable cover of claim one wherein said casing is formed from material to which color or ornamental decoration may be applied.

5. The disposable cover of claim 1 wherein color or design which is not ornamental may be applied to the cover.

6. The disposable cover of claim 1 wherein the disposable cover is formed from bio-degradable material.

7. The disposable cover of claim one wherein the seamless casing has an open end of a dimension when not stretched that is greater than the width of the stethoscope head.
(a) said casing having a width when not stretched that is greater than the width of the stethoscope head
(b) said casing longer than the length of the stethoscope head, stem and adjacent tubing
